(19) United States
(12) Patent Application Publication
(10) Pub. No.: US 2008/0215020 A1
(43) Pub. Date: Sep. 4, 2008

(54) APPARATUSES AND METHODS FOR HEALING WOUNDS

(76) Inventors: WILLIAM H. REEVES, Coral Springs, FL (US); Jonathan Reeves, Coral Springs, FL (US)

Correspondence Address: AKERMAN SENTERFITT, P.O. BOX 3188, WEST PALM BEACH, FL, 33402-3188, US

(21) Appl. No.: 12/017,935

(22) Filed: Jan. 22, 2008

Related U.S. Application Data
(60) Provisional application No. 60/885,977, filed on Jan. 22, 2007.

(51) Int. Cl. A61M 1/00

(52) U.S. Cl. 604/305

(57) ABSTRACT

The invention relates to the development of apparatuses and methods for healing wounds that combine use of a high glycerin-content hydrogel dressing with negative pressure for removing exudate from a wound. In some embodiments, apparatuses and methods further include a photon-emitting device that delivers near infrared stimulation to the wound for further accelerating wound healing. The apparatuses and methods described herein can be used to heal a variety of wounds, including acute wounds, severe burns, orthopedic and traumatic wounds (e.g, flap and meshed graft), skin resurfacing procedure wounds, and neuropathic wounds (e.g., diabetic pressure ulcers). The hydrogel dressing is capable of absorbing substantial amounts of fluids from stimulated wet wounds as well as donating substantial amounts of fluids to dry or necrotic wounds, depending upon the moisture content and nature of the substrate to which it is applied. Results indicate wounds treated with the apparatuses described herein experience fewer complaints of pain, a decrease in healing time, and a significant cost savings.

### CROSS-REFERENCE TO RELATED APPLICATION

The present application claims the priority of U.S. provisional patent application No. 60/885,977, filed Jan. 22, 2007, which is herein incorporated by reference.

### FIELD OF THE INVENTION

The invention relates generally to the fields of medicine and photonics. More particularly, the invention relates to apparatuses and methods for healing wounds.

### BACKGROUND

Wound healing after the loss of the epidermis and exposure of the dermis and other anatomical structures is critical regardless of the modality employed to create the wound, whether laser, deep chemical peel, dermabrasion, burns, surgical incisions or ulcers due to neuropathy. Today, confusing arrays of wound dressings exist, which include totally occlusive (retain all exudate), occlusive yet gas permeable, semi-occlusive (retain some but not all exudate) and gas permeable, and non-occlusive (dry) dressings.

Clinical evidence indicates that maintaining a moist wound environment facilitates the healing process and that a dry wound environment promotes crushing and eschar formation, which impedes keratinocyte migration and slows the healing process. The use of moisture-retentive dressings, dressings which are capable of maintaining a warm, moist environment, appears to accelerate the healing process and promote tissue growth and a number of occlusive dressings have been developed over the past few decades. Various dressing materials have been introduced which consist of film, foam, mesh, or various hydrogel formulations; each with some unique occlusive features which have been useful adjuncts in facilitating wound healing.

Moisture retentive dressings not only keep cells viable, but they enable the cells to release growth factors which promote tissue growth and wound healing. Some growth factors serve to recruit and stimulate different cell types during the wound healing process by, for example, acting on the epidermal cells or keratinocytes as they differentiate to form the outer stratum and aiding the process of angiogenesis by providing a stable collagen matrix. Further, moisture helps wounds heal by facilitating the recruitment of vital host defenses and the necessary cell populations, such as macrophages, which help in the wound healing process. These cells elaborate an amazing number of growth factors, creating a milieu characterized by accelerated angiogenesis, increasing fibrinolysis, and accelerating the rate of healing.

Currently available wound dressings however, are associated with a number of disadvantages. Conventional dry dressings shed fibers into the wound, adhere to the wound base, and dehydrate the wound. Use of low-adherent dressings may require use of a secondary dressing to absorb excess exudate. Hydrocolloid dressings are not suitable for infected wounds. Vapor-permeable adhesive films are suitable only for relatively shallow wounds, while traditional hydrogel dressings are not suitable for use with infected or heavily exuding wounds and most require a secondary dressing. An ideal wound dressing would be one that: is capable of keeping the wound moist, without excessive moisture; is capable of absorbing excessive amounts of wound exudates; exerts a strong bacteriostatic action; is able to remain on the wound for three to seven days, and sufficiently strong to resist the pressure of added weight from fluid accumulation; non-traumatic to the wound bed on removal; is able to keep growth factors supplied in place, i.e. on the wound bed; is able to modulate serious infectious reactions; and is suitable for use with a wide variety of wounds. Thus, a means of dressing a wound that can provide all of these properties would satisfy a great need.

### SUMMARY

The invention relates to the development of apparatuses and methods for healing wounds that combine use of a high glycerin-content hydrogel dressing (also referred to as “high-glycerin content hydrophilic hydrogel dressing”) with negative pressure for removing exudate from a wound. In some embodiments, apparatuses and methods further include a photon-emitting device (e.g., light emitting diode (LED)) that delivers near infrared stimulation to the wound for further accelerating wound healing. The apparatuses and methods described herein can be used to heal a variety of wounds, including acute wounds, severe bums, orthopedic and traumatic wounds (e.g, flap and meshed graft), skin resurfacing procedure wounds, and neuropathic wounds (e.g., diabetic pressure ulcers). The hydrogel dressing is capable of absorbing substantial amounts of fluids from stimulated wet wounds as well as donating substantial amounts of fluids to dry or necrotic wounds, depending upon the moisture content and nature of the substrate to which it is applied. Results indicate wounds treated with this invention experience fewer complaints of pain, a decrease in healing time, and a significant cost savings.

Apparatuses for healing wounds as described herein provide a number of additional advantages for wound healing. They are capable of keeping the wound moist, without excessive moisture retention, while absorbing excessive amounts of wound exudates; the high-glycerin content hydrophilic hydrogel component has strong natural bacteriostatic and fungistatic action; the dressing can remain on the wound surface for three to seven days, the dressing having sufficient strength to resist the pressure of added weight from fluid accumulation; and the hydrogel dressing encourages keratinocyte migration into the wound promoting rapid re-epithelialization and increasing collagen formation in the wound bed all facilitated by the hypoxic environment in the wound bed under the occlusive dressing. Additionally, the dressing is non-traumatic to the wound bed on removal, eliminating wound maceration while keeping the wound growth factors in place beneath the fine glycerin film covering the wound, i.e. on the wound bed while allowing the small water molecules to escape and be captured by the hydrogel. Further, the dressing is able to modulate serious infectious reactions and inflammation, while reducing pain. As the wound re-epithelializes, the non-viable tissue within the wound bed is lifted from the surface of the wound bed by the advancing epithelium, a characteristic not previously reported for any other dressing material. A vacuum pump and system controller are employed to remove the excess wound exudate from the periphery of the dressing as the absorbed wound exudate dissolves the hydrogel and migrates to the edge of the dressing, as well as from the middle of the wound which may be deeper than the edges of the wound.

Accordingly, the invention features an apparatus for healing wounds, the apparatus including a first layer having a first hydrophilic hydrogel substance, the first layer positioned above the wound and in contact with the wound; a first conduit for removing fluid from the wound, the first conduit positioned on top of the first layer and operably connected to a vacuum pump; a second layer including gauze impregnated with a second hydrophilic hydrogel substance, the second layer positioned above the first conduit; and a third layer including a third hydrophilic hydrogel substance, the third layer extending beyond the wound and adhering to the skin surrounding the wound such that the third layer creates a seal between the wound and ambient air. The first, second, and third hydrophilic hydrogel substances can be the same material. The apparatus can further include a second conduit for removing fluid from the wound positioned between the second and third layers, the second conduit operably connected to the vacuum pump. The apparatus can still further include a photon-emitting device positioned on top of the third layer, the photon-emitting device operably connected to a control device. Each of the first, second, and third hydrophilic hydrogel substances can have a glycerin content in the range of about 65% to about 75%, a pH in the range of about 5 to about 6, and bacteriostatic and fungiostatic properties. The first and second conduits can include plastic tubing.

In another aspect, the invention features a method for healing a wound. The method includes the steps of: (a) providing an apparatus including a first layer having a first hydrophilic hydrogel substance, the first layer positioned above the wound and in contact with the wound; a first conduit for removing fluid from the wound, the first conduit positioned on top of the first layer and operably connected to a vacuum pump; a second layer including gauze impregnated with a second hydrophilic hydrogel substance, the second layer positioned above the first conduit; and a third layer including a third hydrophilic hydrogel substance, the third layer extending beyond the wound and adhering to the skin surrounding the wound such that the third layer creates a seal between the wound and ambient air; (b) applying the apparatus to the wound; and (c) applying negative pressure to the wound using the vacuum pump. In the method, the first, second, and third hydrophilic hydrogel substances can be the same material.

In the method, step (c) of applying negative pressure to the wound removes exudate from the wound. The wound can be one of: an acute wound, a severe bum, an orthopedic wound, a traumatic wound, a skin resurfacing procedure wound, and a neuropathic wound. The apparatus can be applied to the wound for at least three contiguous days. Step (b) of applying the apparatus to the wound results in keratinocyte migration into the wound and increases collagen formation in the wound. The method can further include positioning a photon emitting device on top of the third layer, wherein the photon emitting device irradiates the wound. The photon emitting device can be a light emitting diode.

In yet another aspect, the invention includes a kit for healing wounds. A kit includes at least two sheets of hydrophilic hydrogel substance, at least a first conduit for removing fluids from a wound, a suitable amount of gauze impregnated with hydrophilic hydrogel substance, and instructions for use. a second conduit for removing fluids from a wound. In a kit, each of the at least two sheets of hydrophilic hydrogel substance can have a glycerin content in the range of about 65% to about 75%, a pH in the range of about 5 to about 6, and bacteriostatic and fungiostatic properties. The first and second conduits can include plastic tubing.

Unless otherwise defined, all technical terms used herein have the same meaning as commonly understood by one of ordinary skill in the art to which this invention belongs. Although apparatuses, methods and materials similar or equivalent to those described herein can be used in the practice or testing of the present invention, suitable methods and materials are described below. All publications, patent applications, and patents mentioned herein are incorporated by reference in their entirety. In the case of conflict, the present specification, including definitions, will control. The particular embodiments discussed below are illustrative only and not intended to be limiting.

### BRIEF DESCRIPTION OF THE DRAWINGS

FIG. 1 is a cross-sectional view of one embodiment of an apparatus as described herein applied to a wound.

FIG. 2 is a plan view of the apparatus of FIG. 1 applied to a wound.

FIG. 3 is a series of illustrations of a patient having a wound to which an apparatus as described herein is applied.

FIG. 4A is a cross-sectional perspective view of one embodiment of an apparatus as described herein applied to a wound.

FIG. 4B is a cross-sectional perspective view of another embodiment of an apparatus as described herein applied to a wound.

FIG. 5 is a series of photographs of a wound before irradiation treatment, 3 weeks after irradiation treatment had begun, and 6 weeks after irradiation treatment had begun.

### DETAILED DESCRIPTION

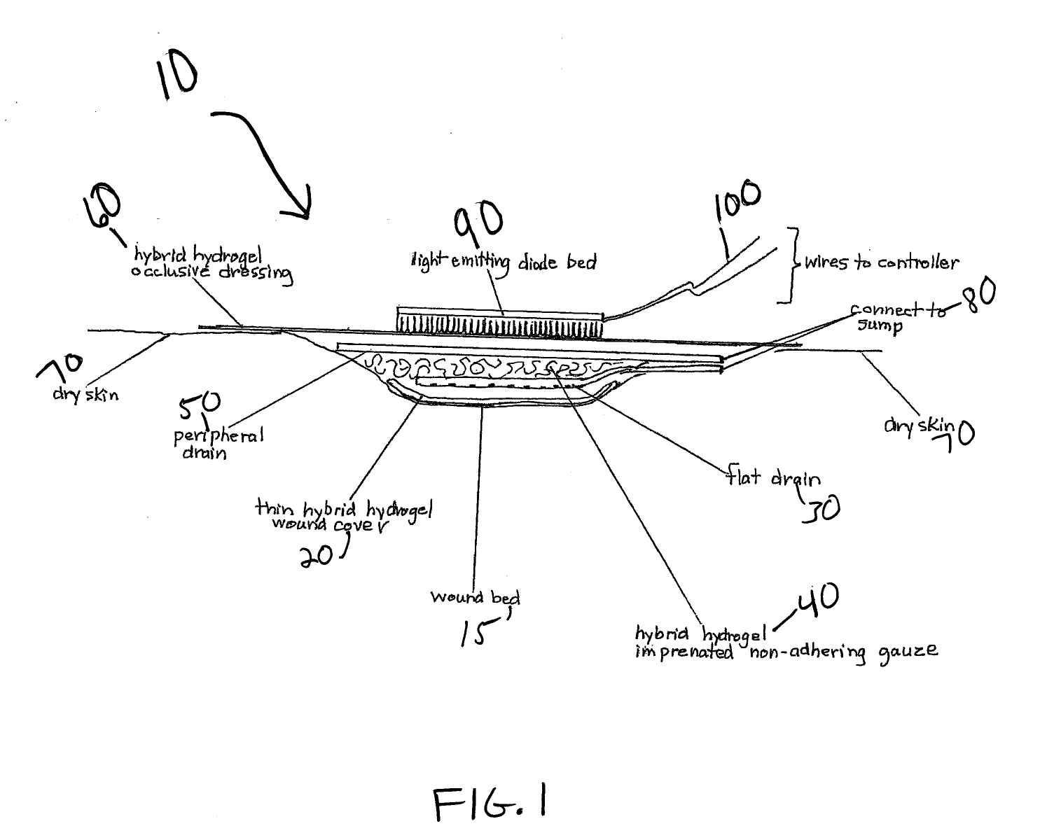

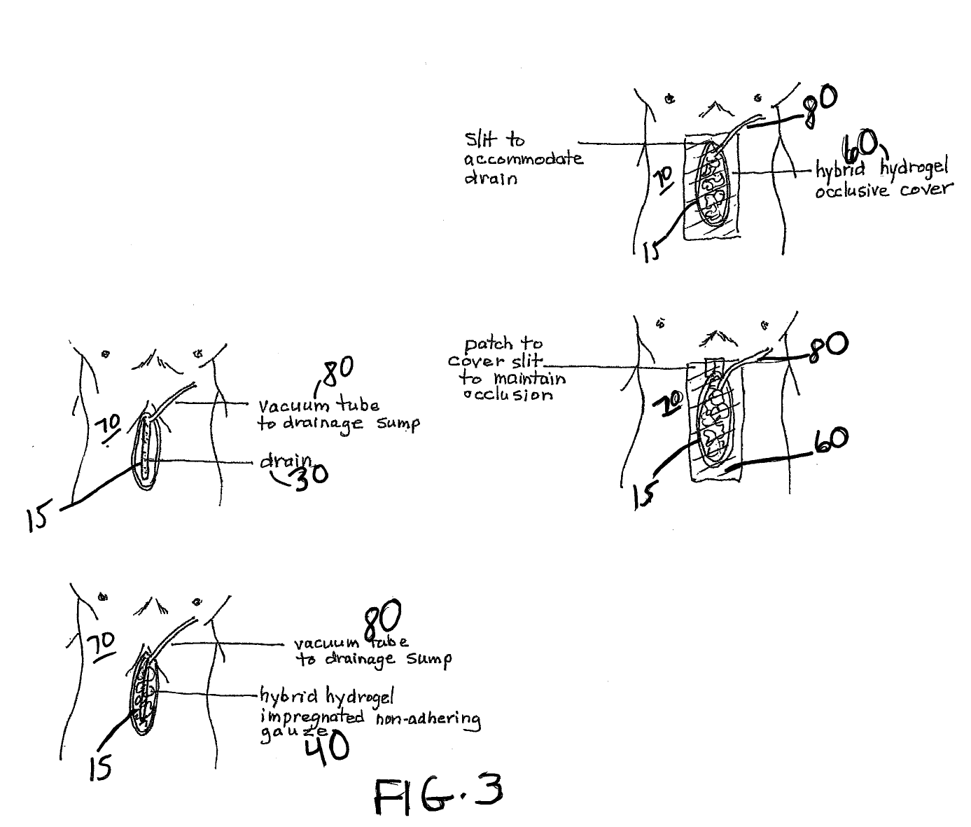

An apparatus for healing wounds as described herein includes a multi-layer dressing including a hydrophilic hydrogel substance containing glycerin and at least one conduit for removing fluid from the wound (e.g., a drain). An apparatus as described herein can be applied to a wound for at least three (e.g., 3, 4, 5, 6, 7, 8) contiguous days. In brief overview, referring to FIGS. 1-3, a first embodiment of an apparatus for healing wounds 10 as described herein is shown applied to a wound bed 15 and includes several layers. A first layer 20 of hydrophilic hydrogel substance is positioned on top of and covering the wound bed 15. First layer 20 is a thin layer of hydrophilic hydrogel substance of any suitable thickness (e.g., 1/20, 1/19, 1/18, 1/17, 1/16, 1/10, ⅛, ⅙, ¼ of an inch), and is typically about 1/16 of an inch. First layer 20 protects the wound bed 15 from direct contact with the first conduit 30 (described below) and provides a moist environment for healing. The hydrophilic hydrogel substance is a blend of glycerin with synthetic hydrophobic polymers in a two-component system in which water enhances the compatibility and function of the dressing and is described in U.S. Pat. No. 5,961,479.

In a typical apparatus, each layer of hydrophilic hydrogel substance has a glycerin content in the range of about 65-70% (e.g., 50%, 55%, 60%, 65%, 70%, 75%, etc.) that contributes to the dressing's positive anti-microbial properties, water soluble humectant in a mixture of water (in the range of about 15-20%) and a polyacrylamide (in the range of about 15-20%). In an example of one embodiment, the hydrophilic hydrogel substance has a glycerin content of approximately 65%, a water content of about 17.5%, and a polyacrylamide content of about 17.5%.

The pH of a hydrophilic hydrogel substance is generally between 5 and 6. Use of the hydrogel provides for the absorbtion of high quantities of fluid (exudate) from the wound. Due to the high percentage of glycerin, the use of hydrophilic hydrogel substances in the apparatuses as described herein confers additional desirable properties, such as bacteriostatic/fungistatic action, retention of growth factors at the wound site, and self-debriding activity, which are absent in conventional hydrogel dressings of high water content.

On top of the first layer 20 is placed a first conduit 30 for removing fluid from the wound. The first conduit 30 is operably connected to a vacuum pump which is operably connected to vacuum pump controller 120, and is typically a drain. The first conduit 30 can be made of any suitable material, e.g., medical-grade plastic tubing, and the dimensions of the first conduit 30 may vary depending on the dimensions of the wound. The vacuum pump and the vacuum pump controller 120 supply suction to the wound bed 15 and at the peripheries of the wound (i.e., wound bed edge 16) to remove the dissolved hydrogel substance as the hydrogel substance removes wound exudates from the wound bed 15 and migrates to the dressing periphery. As the first conduit 30 removes fluids exuding from the wound bed 15 (exudate), it transfers the fluids to a sump collecting chamber 110 or other suitable collection device via tube connector 80. In some embodiments, depending on the type of wound, an apparatus 10 as described herein can further include a second conduit 50 for removing fluids from the wound. When treating wounds such as large bums, traumatic wounds, and diabetic pressure ulcers, for example, an apparatus having a second conduit 50 may be preferable. The second conduit 50 (also referred to as “peripheral drainage tube” or “peripheral drain”) is positioned at the edges of the wound bed 16 or exterior to the wound, extending along the length or circumference of the wound. The second conduit 50 can be made of any suitable material, e.g., medical-grade plastic tubing, and the dimensions of the second conduit 50 may vary depending on the dimensions of the wound. The second conduit 50 is typically perforated plastic tubing having openings along its length for taking up fluids and is also attached to a vacuum pump and sump collecting chamber 110 as is the first conduit 30. As the first layer 20 of hydrogel dressing material absorbs greater amounts of wound exudates, the captured fluids are released in a unique combination of wound exudate and dissolved hydrogel-dressing material, which is collected by the first conduit 30 and at the edges of the wound bed 16 by the second conduit 50.

In a typical apparatus, a second layer 40 of gauze impregnated with hydrophilic hydrogel substance is positioned above the first conduit 30. This second layer 40 packs the wound and provides a moist environment for healing. The gauze can be any suitable gauze for wound dressings, and is combined with any suitable amount of high-glycerin content hydrophilic hydrogel substance (as described in U.S. patent application No. 5,961,479). Gauze provides deposition of a fine film on the wound bed for water molecule transmission to the larger layer of hydrogel above it (i.e., third layer 60 described below). Although gauze is typically used in the apparatuses and dressings described herein, any material that provides a scram or gauze-like texture made of a rectangular weave, coarse cotton or synthetic fiber that will provide a rectangular weave to which the hydrogel material can cling, providing a web-like matrix that can be spread across the wound bed to cover the wound completely, can be used. The next layer of the apparatus 10 is a third layer 60 of hydrophilic hydrogel substance that extends beyond the wound bed 15 and adheres to the skin 70 surrounding the wound, creating a seal between the wound bed 15 and ambient air. The hydrophilic hydrogel substance of third layer 60, of second layer of 40, and of first layer 20 are typically the same material (as described in U.S. Pat. No. 5,961,479), varying generally only in thickness. Different hydrophilic hydrogel substances, however, can be used in the first, second, and third layers of an apparatus if appropriate. Like the other layers containing hydrophilic hydrogel substance, third layer 60 is typically a clear, semi-permeable hydrogel dressing, which can be sized to extend a suitable distance (e.g., 0.5, 1.0, 1.5, 2.0, 2.5 inches) beyond the dimensions of the wound. Third layer 60 protects the adjacent skin, provides an occlusive bond with the skin 70 surrounding the wound, and provides a moist environment for healing. The first conduit 30 is mostly below the third layer 60, but a portion of the first conduit 30 penetrates the third layer 60 and operably connects to tube connector 80 and sump collection chamber 110. To maintain a seal between the wound bed 15 and ambient air, the portion of third layer 60 that surrounds the point of penetration can be pinched together and sealed, or ostomy paste can be placed around the point of penetration to maintain the seal.

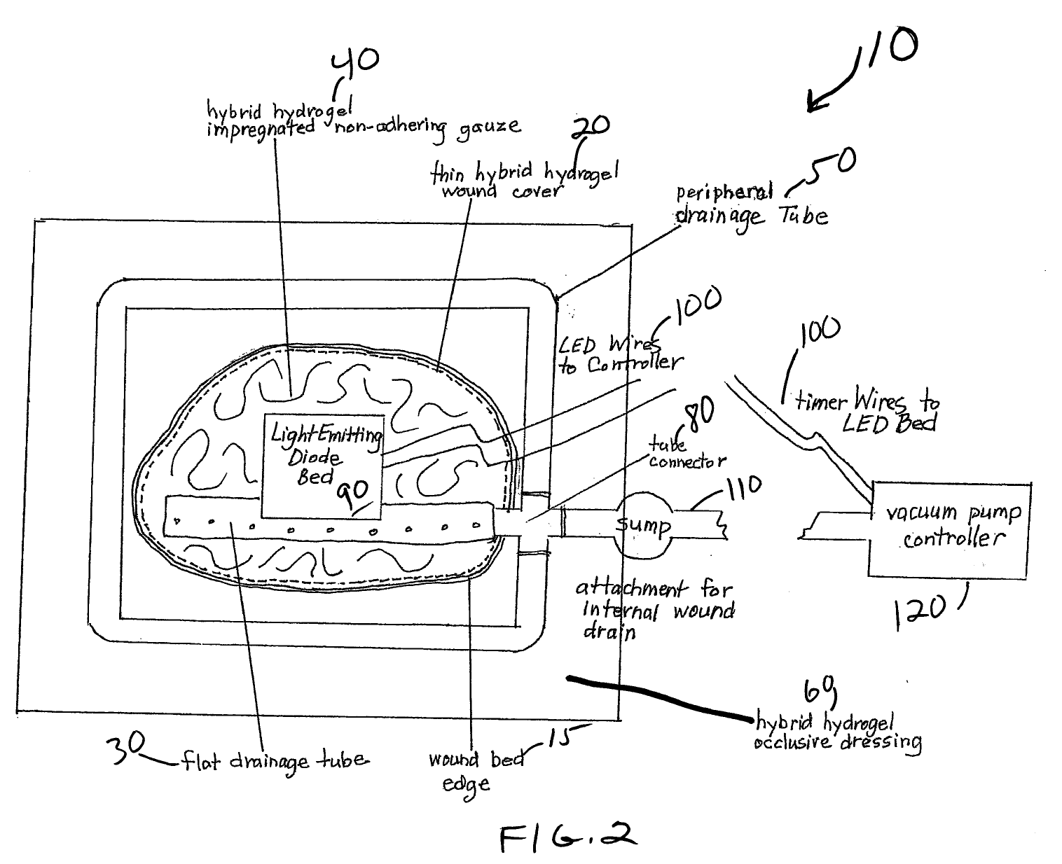

In some embodiments (e.g., FIGS. 1 and 2), an apparatus for healing wounds as described herein further includes a photon-emitting device 90 that is operably connected to a control device. In such embodiments, the photon-emitting device 90 typically provides near infrared light therapy (NILT) to the wound. As wound healing moves through the stages of inflammation, proliferation, remodeling and maturation, NILT can have a positive impact on each of these phases. NILT irradiation can provide a beneficial impact on both closed connective and soft tissue injuries as well as in open wounds in a number of ways. NILT enhances leukocyte (white cell) infiltration to protect the injured tissues. NILT accelerates macrophage activity in phagocytosis (destroying bacteria and foreign material), growth factor secretion (to stimulate tissue repair) and stimulation of collagen synthesis (for tissue integrity), all of these factors contributing to acceleration of healing. Significant angiogenesis (new vessel growth) occurs with NILT promoting new capillary growth resulting in subsequent improvement in blood perfusion and oxygenation. Endothelial cell regeneration is also accelerated.

NILT irradiation stimulates an increase in fibroblast numbers and fibroblast-mediated collagen production. The beneficial synthesis activities and growth factor ability of keratinocytes are enhanced by proliferation secondary to NILT irradiation. NILT-stimulated acceleration of epithelial cell regeneration speeds up wound healing, minimizes scarring and reduces the opportunity for infection. A two- to five-fold increase in growth-phase-specific DNA synthesis in normal fibroblasts, muscle cells, osteoblasts and mucosal epithelial cells irradiated with near infrared light has been reported. This has increased levels of vascular endothelial growth factor (VEGF) and fibroblast growth factor (FGF-2) secondary to NILT. NILT-induced increases in NO, ATP and other compounds that stimulate higher activity in cell proliferation and differentiation cause an increase in mature cells.

The increased numbers of myofibroblasts, myofibrils, and myotubes as well as bone cell proliferation have been clinically documented after NTLT irradiation. Satellite cells, the precursor cells to muscle regeneration, show significant increase in proliferation when irradiated with NILT. Studies have shown that NILT results in greater healed wound tensile strength in both soft tissue and connective tissue injuries. NILT can increase the final tensile strength of the healed tissue by increasing the amount of collagen production/synthesis and by increasing the intra- and inter-molecular hydrogen bonding in the collagen molecules. Studies have also shown that NILT has a significant effect on damaged cells and tissues while normal biological constituents of healthy tissues are appreciably less affected. Methods for using NILT to heal wounds are described, for example, in Rochkind et al., Lasers in Surgery and Medicine 9:174-182 (1989); Braverman et al., Lasers in Surgery and Medicine 9:50-58 (1989); and Surinchak et al., Lasers in Surgery and Medicine 2:267-274 (1983).

When in use, the photon-emitting device 90 is placed directly over the third layer 60 and is connected to a control device via wires 100 or leads. Any suitable photon-emitting device can be used. In a typical embodiment, the photon-emitting device is a LED that provides near infrared stimulation to the wound. Use of a photon-emitting device and NILT in apparatuses and methods described herein is described in greater detail below.

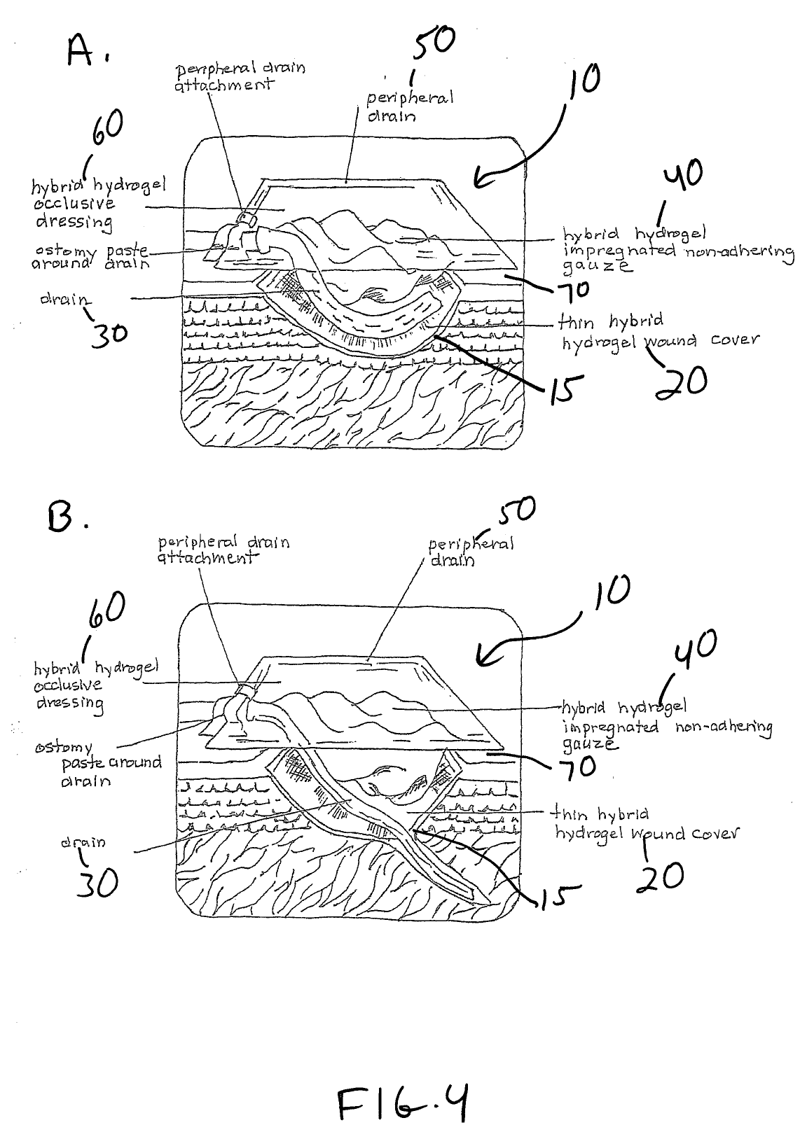

Referring now to FIG. 4, two embodiments of an apparatus for healing wounds 10 are shown. Like the embodiments shown in FIGS. 1-3, the apparatuses of FIG. 4 include a first conduit 30 and a second conduit 50 (e.g., peripheral drain). In FIG. 4A, the apparatus 10 includes a first conduit 30 that is referred to as a “flat drain.” This type of drain is placed on top of the first layer 20. In FIG. 4B, the apparatus 10 includes what is referred to as a “channel drain” which is particularly useful for healing wounds in which an object has penetrated or stabbed the tissue, leaving a deep puncture wound in the wound bed. This type of drain is also placed on top of the first layer 20, but this drain penetrates the first layer 20 and descends into the puncture.

There are several advantages to using an apparatus for healing wounds including a hydrophilic hydrogel substance containing glycerin and at least one conduit for removing fluid from the wound. Due to the high permeability, water content, and pH of the hydrophilic hydrogel dressings used in apparatuses of the invention, pain can be reduced by protecting neurons from dehydration. The cooling effect on wound surfaces may result from the dressing's modulating effect in reducing inflammatory reactions. Other major characteristics of the dressings described herein are elasticity, strength, and durability, which allow the dressing to remain in place for several (e.g., seven) days. As a chemical structure, hydrogels are a three-dimensional network of hydrophilic polymers that interact with aqueous solutions by swelling to certain equilibriums dictated by their different compositions, thus, retaining a significant proportion of water within their structure. Hydrogels are insoluble in water and are non-degradable.

There are additional advantages to using apparatuses and methods described herein that further include a photon-emitting device such as an LED that provides NILT. The photon-emitting device (e.g., LED) provides a visible red or near infrared energy to a wound to promote wound healing (e.g., lymphatic drainage, cellular growth, angiogenesis and tissue migration facilitating closure of the wound). Light therapy has been shown to deliver powerful therapeutic benefits to living tissues. Both visible red and infrared light have shown that they can cause at least 24 different positive changes at the cellular level of tissue. Visible red light, at a wavelength of 630 nanometers (nm), penetrates tissue to a depth of about 8-10 millimeters (mm) and is very beneficial in treating problems close to the surface of the skin. Infrared light (904 nm) can penetrate to a depth of about 30-40 mm, which makes it more effective for treating bones, joints, deep muscle.

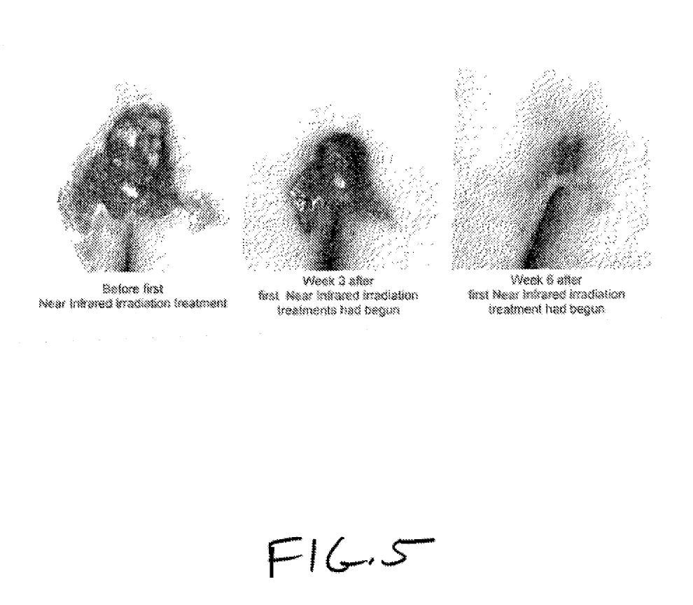

FIG. 5 shows the results of treatment of a wound with NILT. A 660 nm (red) source of 100 joules per cm² was applied to a wound over a ten minute treatment period. An optical probe was used that consisted of an array of 48 monochromatic sources operating at a wavelength of 660 nm and covering an area 6×10 cm². The photographs of FIG. 5 were taken before treatment, 3 weeks after near infrared irradiation, and 6 weeks after near infrared irradiation treatments. In this experiment, 13 week-old pressure sores were treated twice a week for 10 minutes with an LED cluster probe. Two optical probes were used, one consisted of an array of 22 monochromatic sources, operating at a wavelength of 660 nm and covering an area 6×10 cm². The second probe had seven infrared sources, operating at a wavelength of 880 nm and covering an area of 4 cm². Histological inspection showed that infiltration of fibroblasts in the subcutis was significantly greater in the far-infrared (FIR) irradiation group than in the group without FIR irradiation on Days 1, 5, and 7. Furthermore, there was significantly greater collagen regeneration in the FIR group than in the group without FIR on Day 7 in sections with Mallory's staining.

The number of migrated fibroblasts expressing TGF-β1 was significantly greater in the FIR irradiation group, and the production of collagen was increased in the FIR group. The production of collagen fibers due to the activation of fibroblasts by FIR irradiation has been considered as a possible mechanism for the promotive effect of FIR irradiation on wound healing.

The combination of a hydrogel dressing, negative pressure vacuum removal of exudate, and photonic emission (e.g., NILT) provides an ideal environment within which non-healing wounds (e.g., chronic wounds) can begin to re-epithelialize. Use of such an apparatus on a wound promotes angiogenesis, cellular growth, and lymphatic drainage due to the near infrared radiation produced by an array of LEDs and/or laser diodes in several wavelengths coupled with wound healing effects of a high glycerin content containing dressing. Although FIGS. 1 and 2 illustrate a photon-emitting device (e.g., LED display) placed on top of the dressing, radiating through the dressing material to the wound below, in other embodiments, diodes can be incorporated in the hydrogel dressing material. The diodes can be activated and controlled by either a direct electrical connection or by induction from the timer controller, which is either an independent stand-alone control or a module in a negative pressure vacuum pump device. The LED timer typically has multiple settings to control near infrared radiation treatment periods. The combined effects of the near infrared stimulation and the hydrogel dressing can improve wound-healing dynamics of chronic wounds such as pressure sores, diabetic and stasis ulcers, among others, as well as new wounds (e.g., surgical wounds, accidental wounds) and skin flaps.

### Use of Hydrophilic Hyrdogel Substances in Apparatuses for Healing Wounds

Several studies support the fact that the dressings described herein containing hydrophilic hydrogel substances (gels as described in U.S. Pat. No. 5,961,479) do not support growth of any microbe tested but instead kill bacteria that are able to survive on inert surfaces (in such studies, only Bacillus subtilus, a gram-positive rod that can form spores, was not killed). Glycerin in high concentrations has a slight but definite anti-microbial action, which accounts for the way bacterial growth is hampered. It is expected that bacterial size precludes penetration of the gel by bacteria in hydrophilic hydrogel dressings as described herein.

Glycerin also appears to have an immunomodulating effect, which influences the inflammatory response to injury. Cell cultures of human lymphocytes are hampered in their reaction to foreign epidermal cells in the presence of glycerin in even the smallest amounts. Glycerin's strong negative charge binds to extra cellular matrix molecules, modifying their break down and subsequently modulating the inflammatory response.

The bilaminate construction of the hydrophilic hydrogel dressings used in apparatuses and methods as described herein provides a homogeneous hydrogel composite dressing deposited on a mechanically stable substrate, such as a knit like material, which fits comfortably over the wound and creates intimate contact with all surfaces, while protecting and promoting early healing of wound trauma created by laser, chemical deep peelings, derma-abrasion, or delicate plastic/re-constructive or surgical incisions or ulcers. Bilaminate construction helps reduce the normally high-water-vapor transmission rates often associated with hydrogel dressings to much lower, clinically acceptable levels. By maintaining a moist environment the wound dressing decreases the chances of contamination and bacterial infection and initiates immediate pain relief. Mechanically, the layered construction of the apparatuses described herein protects the underlying hydrogel from tearing and puncturing while ensuring conformability to the wound site.

### NILT and Reducing Acute Inflammation and Reducing Pain

Apparatuses and methods for healing wounds as described herein including use of a photon-emitting device that provides NILT can be used to reduce acute inflammation at a wound site. NILT can be effective in mediating the underlying inflammatory process by several actions including: restoring polarity and stability of cellular membrane element concentrations of calcium, sodium and potassium ions as well as the proton gradient over the mitochondria membrane, increasing ATP production and synthesis which contributes to cellular repair, reproduction and function, inducing vasodilation which reduces ischemia (lack of oxygen) and improved perfusion (blood circulation), acceleration of leukocytic (white cell) activity resulting in enhanced removal of non-viable cellular and tissue components and allowing a more rapid repair and revitalization process, reducing the effect of pro-inflammatory cytokines that have been implicated in the development of inflammatory conditions, enhancing the lymphocyte response, and increasing cytokine superoxide dismutase (SOD), a powerful antioxidant, levels.

Since NILT does not exacerbate the inflammatory process but rather condenses the time frame from onset to resolution, it can be used immediately after an injury. This rapid initiation of therapy after acute inflammation occurs can assist in limiting the scope and duration of the inflammatory event and minimize the pain and severity associated with it. As NILT is initiated in more chronic inflammatory conditions, the treatment regimen and course of therapy may be modified by addition of the time required for the desired response, but, the physiological responses and interactions remain consistent.

Apparatuses and methods for healing wounds as described herein can also be used to reduce pain caused by a wound. The unique pain-reducing abilities of NILT irradiation have been researched and documented in numerous clinical studies and medical papers. NILT provides pain relief by several mechanisms, including: increasing b-endorphin production, blocking depolarization of C-fiber afferent nerves, increasing nitric oxide production, increasing nerve cell action potential, nerve cell regeneration and axonal sprouting, decreasing Bradykinin levels, and increasing release of acetylcholine.

### Kits for Healing Wounds

The invention also includes kits for healing wounds. A typical kit includes at least two sheets of hydrophilic hydrogel substance, a suitable amount of gauze impregnated with hydrophilic hydrogel substance, and instructions for use. In some embodiments, a kit can also include one or more conduits for removing fluids. A suitable amount of gauze impregnated with hydrophilic hydrogel substance is an amount sufficient to cover the wound bed. A sheet of hydrophilic hydrogel substance is a layer of hydrophilic hydrogel substance adhered to a non-adherent backing (e.g., a plastic film). In a typical method of using a kit as described herein, the wound is first irrigated thoroughly with 30 ml of saline solution and is patted dry. A first layer of hydrophilic hydrogel substance is removed from its backing and is placed on the wound bed, covering all of the wound. Next, a conduit for removing fluids (e.g., a drain) sized to fit the wound is placed on top of the first layer. Care is taken so that the conduit for removing fluids is not placed such that it is directly contacting the wound or placed into any unexplored fistula tract. After the conduit for removing fluids is positioned, gauze impregnated with hydrophilic hydrogel substance is packed on top of the conduit and the first layer, completely covering the conduit. A third layer of hydrophilic hydrogel substance is then placed over the filled wound, leaving a suitable border (e.g., two inch border) that extends beyond the edges of the wound. Because the conduit penetrates the third layer so that it can be connected to a sump collection chamber and vacuum pump controller, the third layer around the site of penetration is pinched or crimped to create a seal, or a suitable amount of ostomy paste can be placed around the site of penetration to create a seal. An airtight closure is required when negative pressure is applied by the vacuum pump. The conduit can be secured to the sump collection chamber by using adhesive tape. After an airtight seal is created, the vacuum pump controller settings are typically set between 60-80 mm Hg and the vacuum pump is turned on. If the dressing fails to contract when negative pressure is applied, it is not sufficiently sealed. To sufficiently seal the dressing, the area of penetration around the conduit can be reinforced, and/or the connection between the conduit and the sump collection chamber can be reinforced.

### EXAMPLES

The present invention is further illustrated by the following specific examples. The examples are provided for illustration only and should not be construed as limiting the scope of the invention in any way.

### Example 1

### Method of Healing a Wound

In a typical method of healing a wound, a modified Chariker-Jeter dressing technique as described in Chariker et al., (Contemp Surg 34:59-63 (1989)) is employed. The technique involves a thin film of hybrid hydrogel dressing in the base of the wound, a peripheral drainage tube and a flat Jackson-Pratt drain in the center of the wound, with a thin transparent hydrogel film on top of the drain and a connecting tube. An apparatus as described herein is applied to the wound for a period of time sufficient to achieve healing.

In this example, a wound to be healed is covered with a clear, semi-permeable hybrid hydrogel dressing, which is cut to fit two inches beyond the dimensions of the wound, thus protecting the adjacent skin and providing an occlusive bond with the dry skin surrounding the wound. A hydrogel impregnated gauze sandwich is made around the flat, silicone Jackson-Pratt drain. The entire drain sandwich is placed on the wound and covered with another piece of the hybrid hydrogel dressing to create a complete seal. The drain is connected to tubing, which is then connected to the vacuum pump. The movable near infrared LED is placed directly over the wound. The LED head has multiple branches either 77 LEDs in 11 branches of 7 each or for 35 LEDs in 7 branches of 5 each, the latter producing 918 mW or 35×26 mW, if Vishay IRDC are used, driving each with 75 mA. The leads are then attached to a controller which provides a multitude of choices of treatment times and near infrared frequencies.

In this method, the vacuum pump is set at 80 mm Hg of negative pressure in constant mode (Usupov and Yepifanov, Vestnik Khirugii 4:42-45 (1987); Wackenfors et al., Wound Repair Regen 12:600-606 (2004)). When being used at home, the patient can use negative pressure wound therapy (NPWT) for six to eight hours in every 24 hour period. Therapy is administered during night hours when the patient is sleeping. This facilitates normal activities of daily living and is a unique feature of the treatment modality. A memory in the controller will record patient compliance with dates and recorded treatment time. The controller has an automatic timer as a safe guard against treatment time exceeding an acceptable or desirable length.

Using this treatment plan, the patient undergoes NPWT until the healing of the wound plateaus and therapy can be discontinued. When a healthy bed of granulation tissue is identified, the vacuum therapy can be ended. The patient can then apply a composite hybrid hydrogel dressing every four to seven days to promote reepithelialization. Complete healing generally occurs within three weeks and the dressings can be removed.

In another method of healing a wound, the components of the apparatus are applied individually to the wound, rather than a “dressing sandwich” that is applied to the wound. In a first step of this variation of a method, the wound bed is protected by placing a first layer of thin film of hybrid hydrogel substance on the wound bed. Next, a drain is cut to the appropriate length to fit the size of the wound, and the drain is placed over the first layer. Next, the wound is filled or packed with a second layer of gauze that is impregnated with hybrid hydrogel substance. The wound is then covered with a third layer of hybrid hydrogel substance (also referred to as hybrid hydrogel occlusive dressing) leaving a two inch border that extends beyond the wound to ensure a proper occlusive seal. The drain penetrates this third layer and connects to sump collection chamber which is connected to a vacuum pump controller. To create a seal around the area of penetration, the third layer surrounding the penetration site can be pinched to create a seal, or ostomy paste can be applied around the site of penetration to create a seal. If a photon-emitting device is being used, the device (e.g., LED) is next placed on top of the third layer. Then, the near infrared timer and negative pressure controller are adjusted to the appropriate settings and turned on.

### Example 2

### Absorptive Qualities of an Apparatus for Healing Wounds

The absorptive qualities of a hydrophilic hydrogel substance used in methods and apparatuses as described herein were tested in an independent test comparing it to the performance of six leading wound dressing materials (hydrocolloid, membrane, hydrogel and an alginate) over a test period of ninety-six hours. The absorptive properties were measured by evaluating the absorption of a 0.09% saline solution, simulating wound exudate at room temperature. Each dressing was examined at various standard time intervals starting after the first half hour and continuing for a period of ninety-six hours. All but one dressing (an alginate) continued to absorb saline for the entire test period. Within six hours the hydrogel dressing had absorbed eighty-five grams of fluid, out-performing all of the dressings tested.

### Results

The Vigilon™ wound dressing absorbed less than twenty grams of water for the entire ninety-six hours. Clearsite™, Polymem™, and Duoderm™ absorbed less than thirty grams of fluid in the ninety-six hour period. Polyderm™ appeared to reach peak absorption at 0.5 hours. DuoDerm™ began to dissolve after twenty-four hours, so no further measurements were possible. Restore™ absorbed less than seventy grams of fluid in ninety-six hours. At one hour, the invention had absorbed significantly more fluid than any other dressing except Kaltostat™ (alginate), which was expected.

At three hours the hydrogel dressing had absorbed a similar amount of fluid to that of Kaltostat™, but significantly more than all other dressings. At six hours the hydrogel dressing was the only dressing continuing to absorb significant amounts of fluids. None of the test dressings absorbed more than seventy grams of fluid during the ninety-six hour period except the hydrogel dressing which had absorbed 85.0 grams at six hours, 111.2 grams at twelve hours, 26 grams at twenty-four hours, 56.4 grams at thirty-six hours and 173.0 grams at ninety-six hours.

### Example 3

### Heat Shock Protein and Cytokeratin Expression, and Phase S Count in Epidermal Layer at Different Times after Laser Treatment and Dermal Vascular Pattern Characterization

Using a porcine model, back skin was submitted to laser wounds and treated with different occlusive dressings (Flexan, Aquaphor®, Vigilon™ or a hydrophilic hydrogel dressing described herein, either cold or regular (i.e., room temperature)) or left exposed to air. The results showed that all topical treatments epithelized faster than untreated air-exposed skin. Wounds treated with the hydrophilic hydrogel dressing (regular or cold) had a thicker epithelium by day 3 post-treatment than air-exposed skin, suggesting an earlier epithelial maturation time.

### Materials and Methods

Tissue samples: Paraffin blocks from skin used in porcine experiments were used. A total of 4 samples from different animals obtained at day 1 and day 3 post-laser treatment were used for each occlusive dressing. As a control, 4 samples of different animals obtained at the same time, but air-exposed, were used.

4 μm thick sections were obtained from each block. Sections were deparaffinized, and rehydrated. On them, the following determinations were performed:

Low and high molecular weight cytokeratin determination: Deparaffinized sections were incubated during 10 minutes with an endogenous peroxidase blocking agent, washed in saline and then incubated for 30 minutes at room temperature with monoclonal sera of commercial origin directed against Low or High molecular weight cytokeratins (BioGenex). Reaction was revealed with an immunoenzymatic method using a Peroxidase-AntiPeroxidase (P.A.P.) commercial kit (Vectastain Elite Universal Kit, Vector).

Heat Shock protein (hSP) determination: Deparaffinized sections were incubated during 10 minutes with a peroxidase blocking agent, and then incubated for 30 minutes at room temperature with poly or monoclonal sera of commercial origin directed against HSP27 (BioGenex), HSP70, HSP84 and HSP104 (Affinity Bioreagents, Inc). Reaction was revealed with an immunoenzymatic method using a P.A.P. commercial kit (Vectastain Elite Universal Kit, Vector).

Vascular pattern characterization: In order to better characterize the vascular pattern of injured dermis, blood vessels were subdivided in two groups: one of them corresponding to normal dermal plexus and the other, composed of new blood vessels. Differences between these two groups were established by morphological examination and by means of immunoenzymatic determinations of clotting factor VIII distribution pattern and actin in deep endothelial cells. Normal dermal vessel diameters were measured by means of a morphometric program included in an image processor system coupled to the light microscope (Quantimet 500+, Leica Co.). Measurements were performed only on round-shaped vessels, discarding those that were cut in a tangential way. At least 10 determinations were performed per sample, and results were expressed in micrometers (mm) as the mean of these measurements. Neo-formation vessels were counted, referring to an area unit obtained by means of a grid added to the monitor screen, using the same image processor device named before.

Newly formed vessels are immature ones, so they do not express clotting factor VIII in their endothelium nor possess deep endothelial cells (pericytes). By determining the presence of pericytes by actin pattern and the maturation grade of endothelial cells by clotting factor VIII production, one can distinguish between newly formed and already established vessels.

Ploidy measurements: Alternative sections were stained with a modified acid fuchsin method (Feulgen stain). Sections were analyzed in an image processor system (Zeiss) with a densitometry program. At least 40 basal cells from each sample were analyzed. The epidermis immediately neighboring the wound was selected as the area to be analyzed. The program selects and numbers cells with a euploid DNA content and express the value as a deviation index (2cDi) of DNA content of a diploid cell (diploid cell reference used was lymphocytes or dermal cells). Results were expressed as an histogram, where the value 4° C. corresponds to tetraploid cells (those having a double DNA content), indicating that cells finished the S period of the cell cycle.

### Results

Low molecular weight cytokeratin expression: In day 1 samples, no low molecular weight cytokeratin expression could be demonstrated in either group. By day 3, a scanty expression could be observed in the regenerating epithelia from samples treated with Aquaphor Vigilon™, the hydrophilic hydrogel dressing (regular or cold) and from air-exposed skin. Flexan-treated skin showed no expression. Table 1 summarizes the results obtained.

TABLE 1

| | | | | hydroph. | hydroph. | |
|---|---|---|---|---|---|---|
| | | | | hydrogel | hydrogel | Air- |
| | | | | dressing | dressing | ex- |
| | Aquaphor ® | Vigilon ™ | Flexan | Cold | Regular | posed |
| Day | −− | −− | −− | −− | −− | −− |
| 1 | | | | | | |
| Day | + | + | −− | + | + | + |
| 3 | | | | | | |

References
−− No expression;
+ Scanty;
++ Moderate;
+++ strong

Table 1: Low molecular weight cytokeratin expression in CO₂ laser-wounded pig skin treated with different occlusive dressings at day 1 and 3 after laser application.

High molecular weight cytokeratin expression: Using this serum, no differences could be registered in high molecular weight cytokeratin distribution pattern among different samples in these 2 days checked.

HASP expression: In day 1 samples, a weak HSP27 expression was detected on epithelia from all samples. But, by day 3, regular and cold wounds treated with the hydrophilic hydrogel dressing showed a moderate expression on re-epithelization areas, the expression being less in the wounds treated with regular hydrophilic hydrogel dressing than in the wounds treated with cold hydrophilic hydrogel dressing. The remaining samples showed no changes. Other HSPs expression showed no differences among samples. Table 2 summarizes the results obtained.

TABLE 2

| | | | | hydroph. | hydroph. | |
|---|---|---|---|---|---|---|
| | | | | hydrogel | hydrogel | Air- |
| | | | | dressing | dressing | ex- |
| | Aquaphor | Vigilon | Flexan | cold | regular | posed |
| Day | 1 | 1 | 1 | 1 | 1 | 1 |
| 1 | | | | | | |
| Day | 1 | 1 | 1 | 3 | 2 | 1 |
| 3 | | | | | | |

References:
0 absent;
1 minimal;
2 moderate;
3 moderate-marked;
4 marked;
5 exuberant

Table 2: HSP27 expression in CO₂ laser wounded pig skin treated with different occlusive dressings at day 1 and 3 after laser application.

Vascular pattern: By day 1, all samples showed an increase in vascular diameters of blood vessels placed just below the wound, but, by day 3, the mean diameters of Vigilon™, air-exposed and hydrophilic hydrogel dressing (regular and cold)-treated skins increased significantly. Among these, cold hydrophilic hydrogel dressing-treated wounds showed the greatest increase. No changes could be registered in the Aquaphor and Flexan-treated skin.

As expected, by day 3, an angiogenic effect was observed in the wound bed of all samples. Neo-formation vessels could be observed in most samples. 1 out of 4 Aquaphor treated animals showed an important neo-vascularization process. 3 out of 4 Vigilon™-treated animals showed scanty new vessels. Two out of 4 Flexan-treated animals showed scanty new vessels, 3 out of 4 regular hydrophilic hydrogel dressing-treated animals showed a moderate blood vessel neo-formation process. The same occurred in 3 out of 4 cold hydrophilic hydrogel dressing-treated animals. On the contrary, only 2 out of 4 air-exposed animals showed scanty new vessels. Table 3 summarizes the results obtained when vascular diameters were measured. Table 4 summarizes the results obtained when neo-formation vessels were counted.

TABLE 3
Vascular diameters of dermal superficial plexuses, expressed in mm., in
C0₂ laser-wounded pig skin treated with different occlusive
dressings at days 1 and 3 after laser application.

| | | | | | |
|---|---|---|---|---|---|
| | | | hydroph. | hydroph. | |
| | | | hydrogel | hydrogel | |
| | | | dressing | dressing | Air- |
| Aquaphor | Vigilon | Flexan | Cold | Regular | exposed |

Day 1

| | | | | | | |
|---|---|---|---|---|---|---|
| Pig 1 | 0.05 | 0.05 | 0.05 | 0.05 | 0.06 | 0.06 |
| Pig 2 | 0.06 | 0.05 | 0.05 | 0.06 | 0.05 | 0.06 |
| Pig 3 | 0.06 | 0.06 | 0.05 | 0.05 | 0.05 | 0.06 |
| Pig 4 | 0.06 | 0.06 | 0.06 | 0.06 | 0.06 | 0.05 |
| Mean | 0.06 | 0.055 | 0.05 | 0.055 | 0.055 | 0.06 |
| S.D. | 0.00 | 0.01 | 0.00 | 0.01 | 0.01 | 0.00 |

Day 3

| | | | | | | |
|---|---|---|---|---|---|---|
| Pig 1 | 0.06 | 0.10 | 0.06 | 0.11 | 0.10 | 0.08 |
| Pig 2 | 0.07 | 0.08 | 0.05 | 0.09 | 0.09 | 0.08 |
| Pig 3 | 0.07 | 0.08 | 0.06 | 0.11 | 0.08 | 0.07 |
| Pig 4 | 0.06 | 0.09 | 0.06 | 0.11 | 0.10 | 0.08 |
| Mean | 0.065 | 0.09 | 0.06 | 0.105 | 0.09 | 0.08 |
| S.D. | 0.00 | 0.01 | 0.00 | 0.01 | 0.01 | 0.00 |

References:
S.D. standard deviation;
Results are expressed as the mean of at least 10 determinations per animal.

TABLE 4
Number of new blood vessels in CO₂ laser-wounded pig skin treated
with different occlusive dressings at day 3 after laser application. Day 1
data are not shown. Newly formed vessels only became evident at day 3.
Values are expressed as the mean number per area unit. At least 5 areas
per animal were evaluated. An area unit is defined as a 16 mm² area.

| | | | | hydroph. | hydroph. | |
|---|---|---|---|---|---|---|
| | | | | hydrogel | hydrogel | |
| | | | | dressing | dressing | Air- |
| | Aquaphor | Vigilon | Flexan | cold | regular | exposed |
| Pig 1 | 0 | 4 | 0 | 0 | 6 | 0 |
| Pig 2 | 12 | 3 | 6 | 6 | 9 | 4 |
| Pig 3 | 0 | 5 | 4 | 6 | 7 | 0 |
| Pig 4 | 0 | 0 | 0 | 8 | 0 | 4 |
| Mean | 3 | 3 | 2.5 | 5 | 5.5 | 2 |

Ploidy: No significant differences in basal epidermal cell ploidy could be detected among different samples analyzed, neither at day 1 nor at day 3.

### Example 4

### Hydrophilic Hydrogel Substance

A typical hydrophilic hydrogel substance as described herein is a mixture of water, glycerine, and monomeric acrylamide entrapped in a polymer network of cross-linked polyacrylamide and is prepared by mixing the water, glycerine, monomeric acrylamide, initiator (e.g., ammonium persulfate), and methylene bisacrylamide in appropriate amounts under predetermined reaction conditions which initiate reactions and yield the desired properties. An example of a hydrophilic hydrogel substance is below.

| component | amount |
|---|---|
| Glycerine | 66.7% +/− 2% |
| Water | 16.7% +/− 2% |
| Acrylamide* | 16.7% +/− 2% |
| Methylene Bis Acrylamide | 583 ppm** +/− 2% |
| Citric Acid*** | 66 ppm +/− 10% |
| Ammonium persulfate | 33 ppm +/− 10% |

*ppm = parts per million
**Acrylamide can be purchased as a 50% solution and stabilized with a maximum of 25 ppm Cu⁺². The maximum concentration in the product is thus about 8.3 ppm. The acrylamide solutions contains a maximum of 30 ppm of acrylonitrile, most of which reacts to become part of the polymer during the polymerization. An additional reaction occurs on irradiation sterilization.
***Citric Acid is added as a stabilizer for the resulting polymer mixture.

### Other Embodiments

Any improvement may be made in part or all of the components. All references, including publications, patent applications, and patents, cited herein are hereby incorporated by reference. In any listing of possible components, mixtures of possible components are contemplated unless expressly indicated otherwise. The use of any and all examples, or exemplary language (e.g., “such as”) provided herein, is intended to illuminate the invention and does not pose a limitation on the scope of the invention unless otherwise claimed. Any statement herein as to the nature or benefits of the invention or of the preferred embodiments is not intended to be limiting, and the appended claims should not be deemed to be limited by such statements. More generally, no language in the specification should be construed as indicating any non-claimed element as being essential to the practice of the invention. This invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contraindicated by context.

What is claimed is:

1. An apparatus for healing wounds, the apparatus comprising:
(a) a first layer comprising a first hydrophilic hydrogel substance, the first layer positioned above the wound and in contact with the wound;
(b) a first conduit for removing fluid from the wound, the first conduit positioned on top of the first layer and operably connected to a vacuum pump;
(c) a second layer comprising gauze impregnated with a second hydrophilic hydrogel substance, the second layer positioned above the first conduit; and
(d) a third layer comprising a third hydrophilic hydrogel substance, the third layer extending beyond the wound and adhering to the skin surrounding the wound, wherein the third layer creates a seal between the wound and ambient air.

2. The apparatus of claim 1, wherein the first, second and third hydrophilic hydrogel substances are the same material.

3. The apparatus of claim 1, further comprising a second conduit for removing fluid from the wound positioned between the second and third layers, wherein the second conduit is operably connected to the vacuum pump.

4. The apparatus of claim 1, further comprising a photon-emitting device positioned on top of the third layer, wherein the photon-emitting device is operably connected to a control device.

5. The apparatus of claim 1, wherein each of the first, second, and third hydrophilic hydrogel substances have a glycerin content in the range of about 65% to about 75%, a pH in the range of about 5 to about 6, and bacteriostatic and fungiostatic properties.

6. The apparatus of claim 3, wherein the first conduit comprises plastic tubing and the second conduit comprises plastic tubing.

7. A method for healing a wound, the method comprising the steps of:
(a) providing an apparatus comprising: (i) a first layer comprising a first hydrophilic hydrogel substance, the first layer positioned above the wound and in contact with the wound; (ii) a first conduit for removing fluid from the wound, the first conduit positioned on top of the first layer and operably connected to a vacuum pump; (iii) a second layer comprising gauze impregnated with a second hydrophilic hydrogel substance, the second layer positioned above the first conduit; and (iv) a third layer comprising a third hydrophilic hydrogel substance, the third layer extending beyond the wound and adhering to the skin surrounding the wound, wherein the third layer creates a seal between the wound and ambient air;
(b) applying the apparatus to the wound; and
(c) applying negative pressure to the wound using the vacuum pump.

8. The method of claim 7, wherein the first, second and third hydrophilic hydrogel substances are the same material.

9. The method of claim 7, wherein step (c) of applying negative pressure to the wound removes exudate from the wound.

10. The method of claim 7, wherein the wound is selected from the group consisting of: acute wound, severe burn, orthopedic wound, traumatic wound, skin resurfacing procedure wound, and neuropathic wound.

11. The method of claim 7, wherein the apparatus can be applied to the wound for at least three contiguous days.

12. The method of claim 7, wherein step (b) of applying the apparatus to the wound results in keratinocyte migration into the wound and increases collagen formation in the wound.

13. The method of claim 7, further comprising positioning a photon emitting device on top of the third layer, wherein the photon emitting device irradiates the wound.

14. The method of claim 13, wherein the photon emitting device is a light emitting diode.

15. A kit for healing wounds comprising at least two sheets of hydrophilic hydrogel substance, at least a first conduit for removing fluids from a wound, a suitable amount of gauze impregnated with hydrophilic hydrogel substance, and instructions for use.

16. The kit of claim 15, further comprising a second conduit for removing fluids from a wound.

17. The kit of claim 15, wherein each of the at least two sheets of hydrophilic hydrogel substance have a glycerin content in the range of about 65% to about 75%, a pH in the range of about 5 to about 6, and bacteriostatic and fungiostatic properties.

18. The kit of claim 16, wherein the first conduit comprises plastic tubing and the second conduit comprises plastic tubing.
